(19) United States
(12) Patent Application Publication
(10) Pub. No.: US 2006/0162017 A1
(43) Pub. Date: Jul. 20, 2006

(54) Hybrid cotton plants and seeds, and methods and systems of generating same

(76) Inventor: Yechiel Tal, Bat Yam (IL)

Correspondence Address: Martin Moynihan;Anthony Castorina, Suite 207, 2001 Jefferson Davis Highway, Arlington, VA, 22202, US

(21) Appl. No.: 10/541,349

(22) PCT Filed: Jan. 7, 2003
(86) PCT No.: PCT/IL03/00018

§ 371 Date: Jul. 5, 2005

(51) Int. Cl. A01H 1/00; C12N 15/82; A01H 5/00; C12N 5/04

(52) U.S. Cl. 800/279; 800/314; 435/419

(57) ABSTRACT

Novel hybrid cotton plants and seeds are provided herein. Also provided are methods and systems for generating such plants.

### FIELD AND BACKGROUND OF THE INVENTION

The present invention relates to new and distinctive hybrid cotton plants and, more particularly, to hybrid cotton plants designated INTERCOTT-35, INTERCOTT-51, INTERCOTT-75, INTERCOTT-34, INTERCOTT-145, and INTERCOTT-83. The present invention also relates to methods and systems of generating these hybrid cotton plants.

Cotton is an important and valuable field crop which is used to manufacture textile products, oil, animal feed, cordage and other non-woven products. Cotton production today is based mainly on cultivation of varieties of the species Gossypium hirsutum, known as Upland cotton. These cotton varieties are generally preferred for their high lint yield potential, early maturity, and adaptation to adverse climatic and growing conditions. On the other hand, the quality of Upland cotton lint is considered low to medium.

Varieties of another species, G. barbadense, known as Pima cotton, constitute only 5-8% of the world cultivated cotton area. Pima varieties typically produce superior lint having long, strong and fine fiber. On the other hand, these varieties usually have low yield potential, require a long growing season, and can only be cultivated in warm regions.

A primary objective of cotton breeding is to combine in a single cotton variety an improved combination of desirable traits from both species, i.e., the high yield, early maturity and adaptability to diverse growing conditions of the Upland varieties, and the superior fiber qualities of the Pima varieties. However, attempts to transfer genes from one species to another, by way of interspecific crossing and backcrossing, have failed [U.S. Pat. No. 6,102,971; Kohel and Lewis (1984) Cotton Agronomy (series of monographs) Amer. Soc. of Agr. 24: pp 589].

Another approach to obtaining genetic contributions from both species in a single variety is to use the first generation (F1) hybrid as a variety. New hybrid cotton varieties having an improved combination of desired and useful traits can be obtained from a single crossing of pure parental lines of both species, each with different but complimentary desired traits.

Principles of hybrid cotton breeding are described by Davis [Hybrid cotton: specific problems and potentials. (1984) Adv. Agron. 30: 129-1571] and by Anon (1997) [Commercial cotton hybrids. The ICAC Recorder. Vol. XV no. 2: 3-14].

There is thus a widely recognized need for, and it would be highly advantageous to have interspecific hybrids of cotton having a combination of desirable traits derived from the G. Barbadense and G. hirsutum genomes.

### SUMMARY OF THE INVENTION

According to one aspect of the present invention there is provided a hybrid cotton plant, designated as INTERCOTT-35 which is produced by crossing parental lines A-195 and R-208.

Embodiments of this aspect of the present invention also relates to a seed of the hybrid cotton plant; a tissue culture of regenerable cells of the hybrid cotton plant; a tissue culture regenerating plants capable of expressing all the morphological and physiological characteristics of the hybrid cotton plant; and a tissue culture regenerated from cells or protoplasts of a tissue selected from the group consisting of seeds, leaves, stems, pollens, roots, root tips, anthers, ovules, petals, flowers, embryos, fibers and bolls.

According to another aspect of the present invention there is provided a hybrid cotton plant, designated as INTERCOTT-51 which is produced by crossing parental lines A-151 and R-208.

Embodiments of this aspect of the present invention also relates to a seed of the hybrid cotton plant; a tissue culture of regenerable cells of the hybrid cotton plant; a tissue culture regenerating plants capable of expressing all the morphological and physiological characteristics of the hybrid cotton plant; and a tissue culture regenerated from cells or protoplasts of a tissue selected from the group consisting of seeds, leaves, stems, pollens, roots, root tips, anthers, ovules, petals, flowers, embryos, fibers and bolls.

According to yet another aspect of the present invention there is provided a hybrid cotton plant, designated as INTERCOTT-75, which is produced by crossing parental lines A-175 and R-208.

Embodiments of this aspect of the present invention also relates to a seed of the hybrid cotton plant; a tissue culture of regenerable cells of the hybrid cotton plant; a tissue culture regenerating plants capable of expressing all the morphological and physiological characteristics of the hybrid cotton plant; and a tissue culture regenerated from cells or protoplasts of a tissue selected from the group consisting of seeds, leaves, stems, pollens, roots, root tips, anthers, ovules, petals, flowers, embryos, fibers and bolls.

According to still another aspect of the present invention there is provided a hybrid cotton plant, designated as INTERCOTT-34, which is produced by crossing parental lines A-34 and R-208.

Embodiments of this aspect of the present invention also relates to a seed of the hybrid cotton plant; a tissue culture of regenerable cells of the hybrid cotton plant; a tissue culture regenerating plants capable of expressing all the morphological and physiological characteristics of the hybrid cotton plant; and a tissue culture regenerated from cells or protoplasts of a tissue selected from the group consisting of seeds, leaves, stems, pollens, roots, root tips, anthers, ovules, petals, flowers, embryos, fibers and bolls.

According to an additional aspect of the present invention there is provided a hybrid cotton plant, designated as INTERCOTT-145, which is produced by crossing parental lines A-14 and R-205.

Embodiments of this aspect of the present invention also relates to a seed of the hybrid cotton plant; a tissue culture of regenerable cells of the hybrid cotton plant; a tissue culture regenerating plants capable of expressing all the morphological and physiological characteristics of the hybrid cotton plant; and a tissue culture regenerated from cells or protoplasts of a tissue selected from the group consisting of seeds, leaves, stems, pollens, roots, root tips, anthers, ovules, petals, flowers, embryos, fibers and bolls.

According to yet an additional aspect of the present invention there is provided a hybrid cotton plant, designated as INTERCOTT-83, which is produced by crossing parental lines A-83 and R-208.

Embodiments of this aspect of the present invention also relates to a seed of the hybrid cotton plant; a tissue culture of regenerable cells of the hybrid cotton plant; a tissue culture regenerating plants capable of expressing all the morphological and physiological characteristics of the hybrid cotton plant; and a tissue culture regenerated from cells or protoplasts of a tissue selected from the group consisting of seeds, leaves, stems, pollens, roots, root tips, anthers, ovules, petals, flowers, embryos, fibers and bolls.

The present invention also relates to a cotton plant derived from any of the hybrid cotton plants described above, or their parts.

According to another aspect of the present invention there is provided a method of developing a hybrid cotton plant using plant breeding techniques which employ a cotton plant, or its parts, as a source of plant breeding material, the method comprising utilizing cotton plants A-195 and R-208 as a source of breeding material. The method of this aspect of the present invention further relates to plant breeding techniques selected from the group consisting of recurrent selection, backcrossing, pedigree breeding, restriction fragment length polymorphism enhanced selection, genetic marker enhanced selection, and transformation.

According to yet another aspect of the present invention there is provided a method of developing a hybrid cotton plant using plant breeding techniques which employ a cotton plant, or its parts, as a source of plant breeding material, the method comprising utilizing cotton plants A-151 and R-208 as a source of breeding material. The method of this aspect of the present invention further relates to plant breeding techniques selected from the group consisting of recurrent selection, backcrossing, pedigree breeding, restriction fragment length polymorphism enhanced selection, genetic marker enhanced selection, and transformation.

According to still another aspect of the present invention there is provided a method of developing a hybrid cotton plant using plant breeding techniques which employ a cotton plant, or its parts, as a source of plant breeding material, the method comprising utilizing cotton plants A-175 and R-208 as a source of breeding material. The method of this aspect of the present invention further relates to plant breeding techniques selected from the group consisting of recurrent selection, backcrossing, pedigree breeding, restriction fragment length polymorphism enhanced selection, genetic marker enhanced selection, and transformation.

According to an additional aspect of the present invention there is provided a method of developing a hybrid cotton plant using plant breeding techniques which employ a cotton plant, or its parts, as a source of plant breeding material, the method comprising utilizing cotton plants A-34 and R-208 as a source of breeding material. The method of this aspect of the present invention further relates to plant breeding techniques selected from the group consisting of recurrent selection, backcrossing, pedigree breeding, restriction fragment length polymorphism enhanced selection, genetic marker enhanced selection, and transformation.

According to yet an additional aspect of the present invention there is provided a method of developing a hybrid cotton plant using plant breeding techniques which employ a cotton plant, or its parts, as a source of plant breeding material, the method comprising utilizing cotton plants A-14 and R-205 as a source of breeding material. The method of this aspect of the present invention further relates to plant breeding techniques selected from the group consisting of recurrent selection, backcrossing, pedigree breeding, restriction fragment length polymorphism enhanced selection, genetic marker enhanced selection, and transformation.

According to yet an additional aspect of the present invention there is provided a method of developing a hybrid cotton plant using plant breeding techniques which employ a cotton plant, or its parts, as a source of plant breeding material, the method comprising utilizing cotton plants A-83 and R-208 as a source of breeding material. The method of this aspect of the present invention further relates to plant breeding techniques selected from the group consisting of recurrent selection, backcrossing, pedigree breeding, restriction fragment length polymorphism enhanced selection, genetic marker enhanced selection, and transformation.

According to another aspect of the present invention there is provided a system for developing a hybrid cotton plant using plant breeding techniques, the system comprising cotton plants A-195 and R-208 or parts of the cotton plants as a source of the breeding material. Preferably, at least one of the cotton plants is male sterile and female fertile.

According to yet another aspect of the present invention there is provided a system for developing a hybrid cotton plant using plant breeding techniques, the system comprising cotton plants A-151 and R-208 or parts of the cotton plants as a source of the breeding material. Preferably, at least one of the cotton plants is male sterile, preferably characterized by a G. harknesii cytoplasm.

According to still another aspect of the present invention there is provided a system for developing a hybrid cotton plant using plant breeding techniques, the system comprising cotton plants A-175 and R-208 or parts of the cotton plants as a source of the breeding material. Preferably, at least one of the cotton plants is male sterile preferably characterized by a G. harknesii cytoplasm.

According to an additional aspect of the present invention there is provided a system for developing a hybrid cotton plant using plant breeding techniques, the system comprising cotton plants A-34 and R-208 or parts of the cotton plants as a source of the breeding material. Preferably, at least one of the cotton plants is male sterile preferably characterized by a G. harknesii cytoplasm.

According to yet an additional aspect of the present invention there is provided a system for developing a hybrid cotton plant using plant breeding techniques, the system comprising cotton plants A-14 and R-205 or parts of the cotton plants as a source of the breeding material. Preferably, at least one of the cotton plants is male sterile preferably characterized by a G. harknesii cytoplasm.

According to yet an additional aspect of the present invention there is provided a system for developing a hybrid cotton plant using plant breeding techniques, the system comprising cotton plants A-83 and R-208 or parts of the cotton plants as a source of the breeding material. Preferably, at least one of the cotton plants is male sterile preferably characterized by a G. harknesii cytoplasm.

According to another aspect of the present invention there is provided a cotton plant characterized by a combination of traits leading to a commercial yield higher than at least one parent of the cotton plant under growth conditions, selected from the group consisting of suboptimal water supply, high level salinity (6-8 EC), suboptimal temperature, suboptimal light, a growth cycle period shorter than 170 days to full maturity (typically measured as opening of 95% of the bolls), and infestation of pathogens. This aspect of the invention further relates to pathogens selected from the group consisting of Verticillium spp., Fusarium spp., and Alternaria spp.

According to further features in preferred embodiments of the invention described bellow the cotton plant is hybrid cotton plant INTERCOTT-35 and its parents are cotton plants A-195 and R-208.

According to still further features in the described preferred embodiments the cotton plant is hybrid cotton plant INTERCOTT-51 and its parents are cotton plants A-151 and R-208.

According to still further features in the described preferred embodiments the cotton plant is hybrid cotton plant INTERCOTT-75 and its parents are cotton plants A-175 and R-208.

According to still further features in the described preferred 5 embodiments the cotton plant is hybrid cotton plant INTERCOTT-34 and its parents are cotton plants A-34 and R-208.

According to still further features in the described preferred embodiments the cotton plant is hybrid cotton plant INTERCOTT-145 and its parents are cotton plants A-14 and R-205.

According to still further features in the described preferred embodiments the cotton plant is hybrid cotton plant INTERCOTT-83 and its parents are cotton plants A-83 and R-208.

According to another aspect of the present invention there is provided a planted field comprising cotton plants A-195 and R-208.

According to yet another aspect of the present invention there is provided a planted field comprising cotton plants A-151 and R-208.

According to still another aspect of the present invention there is provided a planted field comprising cotton plants A-175 and R-208.

According to an additional aspect of the present invention there is provided a planted field comprising cotton plants A-34 and R-208.

According to yet an additional aspect of the present invention there is provided a planted field comprising cotton plants A-14 and R-205.

According to still an additional aspect of the present invention there is provided a planted field comprising cotton plants A-83 and R-208.

According to still further features in the described preferred embodiments at least one of the cotton plants of the planted field is male sterile.

According to still further features in the described preferred embodiments the field is characterized by a planting pattern enabling cross pollination between the two cotton plants described above, preferably aided by insects such as honey bees or bumble bees or performed manually.

According to a further aspect of the present invention there is provided a method of developing a hybrid cotton plant using plant breeding techniques, the method comprising utilizing a first cotton plant selected from the group consisting of A-195, A-151, A-175, A-34, A-14 and A-83, and a second cotton plant selected from the group consisting of R-205 and R-208, as sources of breeding material.

According to yet a further aspect of the present invention there is provided a system for developing a hybrid cotton plant using plant breeding techniques, the system comprising utilizing a first cotton plant selected from the group consisting of A-195, A-151, A-175, A-34, A-14 and A-83, and a second cotton plant selected from the group consisting of R-205 and R-208, as sources of breeding material.

The present invention successfully addresses the shortcomings of the presently known configurations by providing hybrid cotton plants and methods and systems of generating same, which cotton plants are characterized by a combination of highly desirable traits.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a chart illustrating the breeding process and development of the hybrid cotton plants of the present invention.

### DESCRIPTION OF THE PREFERRED EMBODIMENTS

The present invention is of hybrid cotton plants and methods of production thereof. The plants are developed from a single interspecific crossing between a male sterile Gossypium hirsutum female parent line and a restorer Gossypium barbadense male parent line, and have superior characteristics such as excellent lint quality, high yield, stress tolerance and disease resistance.

The principles and operation of the present invention may be better understood with reference to the accompanying drawing and descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Terminology

As used herein the term “line” and the term “genome” are used interchangeably to refer to the genetic complement contained in the plant.

As used herein the term “hybrid” refers to a first generation progeny (F1) of a cross between two different homozygous parental lines.

As used herein the phrase “interspecific hybrid” refers to the progeny of a cross between two different homozygous parental lines of two different species.

As used herein the term “tolerance” refers to the ability of a plant to endure stress conditions without suffering serious crop loss.

As used herein the term “salinity” refers to soil which contains sufficient soluble salt to adversely affect plant growth. Many agricultural soils, in regions of low rainfall, are high in soluble salts. This condition is usually due to the fact that irrigation waters are high in soluble salt, or often accumulate salts in the watershed from which they are derived due to high evaporation. As the water is applied to crop lands, salts are not leached out as fast as they are applied.

As used herein the phrase “tolerance to salinity” refers to the ability of a plant to endure the effect of salinity without suffering serious crop loss. Salinity tolerant varieties can be cultivated in marginal saline soils, thereby enabling substantial expansion of land use.

As used herein the phrase “tolerance to suboptimal water supply” refers to the ability of a plant to endure the effect of drought or water shortage without suffering serious crop loss. Tolerant varieties which overcome the damaging effect of drought, can be cultivated in arid or semi-arid regions or when irrigation water is limited

As used herein the phrase “tolerance to suboptimal temperature” refers to the ability of a plant to endure the effect of unfavorable growth temperature without suffering serious crop loss. Varieties which can be cultivated in temperate climate areas enable substantial expansion of land use. In addition, varieties which can tolerate lower than optimal soil temperature also enable an early sowing of spring crops.

As used herein the term “resistance” refers to the ability of a plant to exclude or overcome the effect of a pathogen. Plant pathogens may cause diseases resulting in serious crop losses. Varieties resistant to pathogens provide an effective, inexpensive and ecologically beneficial approach to controlling plant pathogens.

As used herein the phrase “full maturity” refers to the growth stage when cotton plants reach an average of 90-100% open bolls.

As used herein the phrase “fiber length” refers to the 2.5% span length (in inches) of fiber as measured by High Volume Instrumentation (HVI).

As used herein the phrase “fiber strength” refers to the force required to break a bundle of fibers as measured in grams per tex on the HVI.

As used herein the phrase “fiber fineness” refers to the fiber perimeter at maturity as measured in micronaire values ranging from about 2.0 (very fine) to 6.0 (very course).

Cotton lint quality is measured according to fiber length, strength and fineness. Accordingly, the lint quality is considered higher when the fiber is longer, stronger and finer when the fiber is fully matured in open boll.

Cotton is an important and valuable field crop. Thus, a primary goal of cotton breeding is to select and develop plants that have the traits that result in superior varieties. It is estimated that 97% of the world production of cotton is generated from varieties of two species, Gossypium hirsutum (Upland) and G. barbadense (Pima). Upland cotton varieties are characterized by having relatively high yield potential, tolerance to adverse climatic and growth conditions. Yet, the lint produced from Upland varieties is of relatively low to medium quality. On the other hand, Pima cotton varieties are characterized by having less yield potential than the Upland and by not being adaptable to adverse climatic or growing conditions. Yet, the quality of lint produced from Pima varieties is considered high to excellent. Hence it is highly desired to combine the economically advantageous traits from the two different species in a single cotton variety.

Thus, according to one aspect of this invention there is provided a cotton plant designated INTERCOTT-35, representative seed of the cotton plant having been deposited under ATCC Patent Depository No.: PTA-4832.

As illustrated in the Examples section which follows, INTERCOTT-35 has numerous important morphological and physiological characteristics including a semi-erect growth habit, long fruit branches, large dark-green leaves, tipped oval shaped boll, an average number of 4-5 locules per boll, averaging 140 days to reach 50 percent open bolls, an average number of 6 nodes before the first fruit branch (i.e. number of nodes present on main stem under the first fruit bearing branch), plant height of 110-130 cm at full maturity, medium pubescence of leaf and stem, presence of gossypol glands, presence of nectaries in flowers and leaves, light yellow petals, presence of flower petal spot, yellow flower pollen, seed index of 13.0 gram per 100 seeds, a light fuzzy seed coat, lint content of 37 per cent, boll seed weight average of 4.5 gr, fiber length average of 1.34 inches, fiber strength average of 36-38 gram per tex and fiber fineness of 3.7-3.9 micronaire, tolerance to suboptimal water supply, and salinity; and resistance to Fusarium wilt, to Verticillium wilt, and to Alternaria leaf spot.

As is mentioned hereinabove, INTERCOTT-35 was generated by crossing female parental line A-195 and male parental line R-208. The characteristic traits of these parental lines are provided in Tables 1 and 2 of the Examples section that follows.

A comparison of INTERCOTT-35 to its parental lines (see, Examples section), demonstrates that this interspecific hybrid exhibits several economically and agronomically advantageous traits over its respective parental lines. In particular, Verticillium wilt resistance (compared with sensitive A-195), fiber fineness of 3.7-3.9 micronaire (compared with 4.3 micronaire of A-195), fiber strength of 36-38 gram per tex (compared with 27.5 and 33 micronaire of A-195 and R-208, respectively), lint percent of 37 (compared with 36.5 and 35 percent, of A-195 and R-208, respectively), and having short or light fuzzy seed coat enabling ginning by either roller or saw systems. In addition, INTERCOTT-35 is tolerant to suboptimal supply of water and to salinity.

Seeds of the hybrid cotton plant of this aspect of the present invention can be generated using breeding and selection techniques. For example, screening techniques such as restriction fragment length polymorphism selection or genetic marker selection, can be employed in combination with recurrent selection, pedigree breeding, transformation and/or backcrossing to generate the most suitable parental lines used for hybrid seed production.

The goal of backcrossing is to alter or substitute a single trait or characteristic in a recurrent parental line. To accomplish this, a single gene of the recurrent parental line is substituted or supplemented with the desired gene from the nonrecurrent line, while retaining essentially all of the rest of the desired genes, and therefore the desired physiological and morphological constitution of the original line. The choice of the particular nonrecurrent parent will depend on the purpose of the backcross. One of the major purposes is to add some commercially desirable, agronomically important trait to the plant. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred. Likewise, transgenes can be introduced into the plant using any of a variety of established transformation methods well-known to persons skilled in the art, such as: Gressel (1985) [Biotechnologically Conferring Herbicide Resistance in Crops: The Present Realities, In Molecular Form and Function of the plant Genome, L van Vloten-Doting, (ed.), Plenum Press, New York]; Huftner, S. L., et al. (1992) [Revising Oversight of Genetically Modified Plants. Bio/Technology]; Klee, H., et al. (1989) [Plant Gene Vectors and Genetic Transformation: Plant Transformation Systems Based on the use of Agrobacterium tumefaciens, Cell Culture and Somatic Cell Genetics of Plants]; and Koncz, C., et al. (1986) [Molecular and General Genetics]. Techniques for transforming cotton plants are described in Umbeck et al. (1987) [Bio/Technology 5:263-266]; Firoozabady et al. (1987) [Plant Mol. Biol. 10:105-116]; Finer and McMullen (1990) [Plant Cell Rep. 8:586-589]; Bayley et al. (1992) [Theo. Appl. Genet. 83:45-649]; Perlak et al. (1990) [Bio/Technology 8:939-943]; and U.S. Pat. Nos. 5,986,181; 5,846,797.

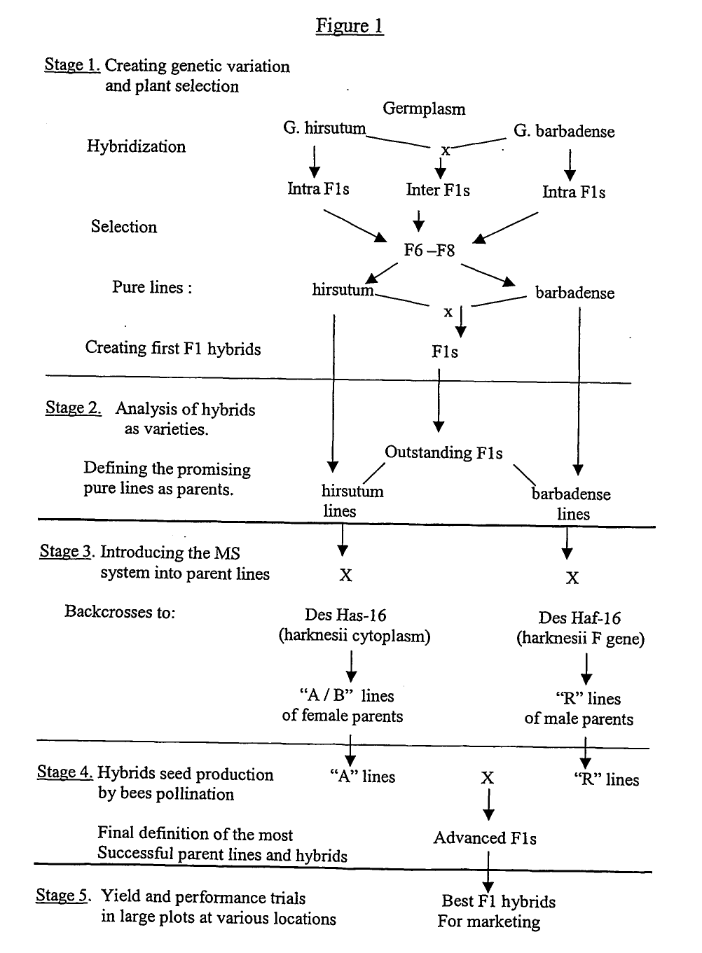

As is illustrated in FIG. 1 and described in the Examples section which follows, seeds of the cotton plant INTERCOTT-35 were generated using a breeding process which began with the generation of highly specific parental lines A-195 and R-208. Unless stated otherwise, plant growth conditions used for growing the parental and hybrid plants of the present invention are as described in the Example section which follows.

The breeding process initiates with a selection of G. hirsutim and G. barbadense plants suitable for initial breeding crosses. These parental lines are backcrossed for 6-8 generations to produce a series of pure lines of each species, which, although different from each other, breed true and are highly uniform.

In a next breeding step, male sterility (MS) is introduced into one of the selected parental lines. The incorporation of MS is necessary in order to facilitates large scale production of hybrid seeds.

There are several approaches for controlling male fertility available to breeders, including manual or mechanical emasculation (or detasseling), cytoplasmic male sterility, gematocides, or genetic male sterility [Meyer (1973) Registration of sixteen germplasm lines of Upland cotton Crop. Sci. 13:778].

According to one embodiment of this aspect of the invention, selected pure lines of G. hirsutum are backcrossed, as recurrent lines, with the MS breeding line DES HAMS-16 (Meyer, 1973), until male sterility is fully incorporated. Once completed, the male sterile lines of G. hirsutum are designated “A” lines and are used as female parental lines, while the respective recurrent original fertile lines are designated “B” lines and are used as maintainer lines. Subsequently, the male sterile “A” lines are essentially identical to their respective maintainer “B” lines, except for the male sterility trait. A description of preferred parental lines of G. hirsutum is provided in Table 1 of the Example section that follows.

On the other hand, selected pure lines of G. barbadense are backcrossed with the MF breeding line DES HAF-16 (Meyer, 1973), until the dominant gene for fertility restoration is fully incorporated. Once completed, the G. barbadense lines capable of fertility restoration are designated “R” lines and were used as male parental lines. A description of preferred parental lines of G. barbadense is provided in Table 2 of the Examples section that follows.

In the final step of the breeding process a single cross is made between a selected parental “A” (female) line of G. hirsutum, and a selected “R” restorer line of G. barbadense, to produce the interspecific hybrid progeny. The cross between two different pure (homozygous) lines produces a uniform population of hybrid plants that may be heterozygous for many gene loci. An important consequence of the homozygosity and homogeneity of the parental lines is that the hybrid created by crossing a defined pair of parental lines will always be the same. Accordingly, the first interspecific hybrids are analyzed and selected for desired traits. Once the parental lines that create a superior hybrid have been identified, a continual supply of the hybrid seed can be produced using these parental lines and the hybrid cotton plants can then be generated from this hybrid seed supply.

While reducing the present invention to practice, it was observed that plants of the second generation (F2) of the interspecific hybrids of the present invention are characterized by high trait variation among individuals of the F2 progeny. In fact, the variation range exceeded that encompassing both inbred parent lines. However, such variation resulted in a progeny which included undesired genotypes.

Once established, INTERCOTT-35 can be propagated from hybrid seeds or alternatively by using tissue culturing techniques.

As used herein the phrase “tissue culture” refers to plant cells or plant parts from which cotton plants can be generated, including plant protoplasts, plant cali, plant clumps, and plant cells that are intact in plants, or part of plants, such as seeds, leaves, stems, pollens, roots, root tips, anthers, ovules, petals, flowers, embryos, fibers and bolls.

Techniques of generating plant tissue culture and regenerating plants from tissue culture are well known in the art. For example, such techniques are set forth by Vasil (1984) [Cell Culture and Somatic Cell Genetics of Plants, Vol I, II, III Laboratory Procedures and Their Applications Academic Press, New York]; Green et al. (1987) [Plant Tissue and Cell Culture, Academic Press, New York]; Weissbach and Weissbach (1989) [Methods for Plant Molecular Biology, Academic Press]; Gelvin et al. (1990) [Plant Molecular Biology Manual, Kluwer Academic Publishers]; Evans et al. (1983) [Handbook of Plant Cell Culture, MacMillian Publishing Company, New York]; and Klee et al. (1987) [Ann. Rev. of Plant Phys. 38:467-486].

The tissue culture can be generated from cells or protoplasts of a tissue selected from the group consisting of seeds, leaves, stems, pollens, roots, root tips, anthers, ovules, petals, flowers, embryos, fibers and bolls. Techniques of generating cotton plant tissue culture and regenerating cotton plants from tissue culture are described, for example, by Umbeck et al. (1987) [Bio/Technology 5:263-266]; Firoozabady et al. (1987) [Plant Mol. Biol. 10:105-116]; Finer J. (1988) [Plant Cell Rep. 6:231-234]; and U.S. Pat. Nos. 5,986,181; 5,846,797.

Thus, this aspect of the present invention provides novel hybrid cotton plants, seeds and tissue culture for generating same. This aspect of the present invention further provides a system for developing such a hybrid cotton plant which system includes cotton plants A-195 and R-208 or parts of thereof as a source of the breeding material.

According to another aspect of the present invention, there is provided a cotton plant designated INTERCOTT-51, representative seed of the cotton plant having been deposited under ATCC Patent Depository No.: PTA-4833.

As is illustrated in the Examples section which follows, INTERCOTT-51 has numerous important morphological and physiological characteristics including a medium-open growth habit, long fruit branches, large light-green leaves, tipped oval shaped boll, an average number of 4-5 locules per boll, averaging 130 days to reach 50 percent open bolls, an average node number of 5.5 before the first fruit branch, plant height of 100-110 cm at full maturity, sparse pubescence of leaf and stem, presence of gossypol glands, presence of nectaries in flowers and leaves, light yellow petals, presence of flower petal spot, yellow flower pollen, seed index of 12.5 gram per 100 seeds, a light fuzzy seed coat, lint content of 36.5 per cent, fiber length average of 1.33 inches, fiber strength average of 32-34 gram per tex and fiber fineness of 3.7-3.9 micronaire, tolerance to suboptimal water supply; and resistance to Fusarium wilt, to Verticillium wilt, and to Alternaria leaf spot.

As is mentioned hereinabove, INTERCOTT-51 was generated by crossing female parental line A-151 and male parental line R-208. The characteristic traits of these parental lines are provided in Tables 1 and 2 of the Examples section that follows.

A comparison of INTERCOTT-51 to its parental lines, demonstrates that this interspecific hybrid exhibits several economically and agronomically advantageous traits over its respective parental lines. In particular, Verticillium wilt resistance (compared with sensitive A-151), fiber length of 1.33 inches (compared with 1.13 and 1.25 inches of A-151 and R-208, respectively), fiber strength of 32-34 gram per tex (compared with 28 and 33 micronaire of A-151 and R-208, respectively), and having short or light fuzzy seed coat enabling ginning by either roller or saw systems after adjusting (reducing) the process speed. In addition, INTERCOTT-51 is characterized with concentrated yield and with tolerance to suboptimal water supply such as under limited irrigation conditions.

Seeds of the hybrid cotton plant of this aspect of the present invention can be generated using breeding and selection techniques as described hereinabove. Accordingly, seeds of the cotton plant INTERCOTT-51 were generated using a breeding process which began with the generation of highly specific parental lines A-151 and R-208, as described hereinabove and as is illustrated in FIG. 1 and in the Examples section which follows.

Once established, INTERCOTT-51 can be propagated from hybrid seeds or alternatively by using tissue culturing techniques, as described hereinabove. Thus, this aspect of the present invention provides novel hybrid cotton plants, seeds and tissue culture for generating same. This aspect of the present invention further provides a system for developing such a hybrid cotton plant which system includes cotton plants A-151 and R-208 or parts of thereof as a source of the breeding material.

According to another aspect of the present invention, there is provided a cotton plant designated INTERCOTT-75, representative seed of the cotton plant having been deposited under ATCC Patent Depository No.: PTA-4834.

As is illustrated in the Examples section which follows, INTERCOTT-75 has numerous important morphological and physiological characteristics including a short-open growth habit, medium fruit branches, medium light-green leaves, tipped oval shaped boll, an average number of 4 locules per boll, averaging 120 days to reach 50 percent open bolls, an average number of 5 nodes before the first fruit branch, plant height of 90-100 cm at full maturity, medium pubescence of leaf and stem, presence of gossypol glands, presence of nectaries in flowers and leaves, light yellow petals, presence of flower petal spot, yellow flower pollen, seed index of 12 gram per 100 seeds, a light fuzzy seed coat, lint content of 36 per cent, fiber length average of 1.32 inches, fiber strength average of 31-33 gram per tex and fiber fineness of 3.8-3.9 micronaire; and resistance to Fusarium wilt, to Verticillium wilt, and to Alternaria leaf spot.

As is mentioned hereinabove, INTERCOTT-75 was generated by crossing female parental line A-175 and male parental line R-208. The characteristic traits of these parental lines are provided in Tables 1 and 2 of the Examples section that follows.

A comparison of INTERCOTT-75 to its parental lines, demonstrates that this interspecific hybrid exhibits several economically and agronomically advantageous traits over its respective parental lines. In particular, Verticillium wilt resistance (compared with sensitive A-175), fiber length of 1.32 inches (compared with 1.12 and 1.25 inches of A-75 and R-208, respectively), and having short or light fuzzy seed coat enabling ginning by either roller or saw systems. In addition, INTERCOTT-75 is characterized by being adapted for a short season requiring just 120 days to achieve maturity of 50 percent open boll.

Seeds of the hybrid cotton plant of this aspect of the present invention can be generated using breeding and selection techniques as described hereinabove. Accordingly, seeds of the cotton plant INTERCOTT-75 were generated using a breeding process which began with the generation of highly specific parental lines A-175 and R-208, as described hereinabove and as is illustrated in FIG. 1 and in the Examples section which follows.

Once established, INTERCOTT-75 can be propagated from hybrid seeds or alternatively by using tissue culturing techniques, as described hereinabove. Thus, this aspect of the present invention provides novel hybrid cotton plants, seeds and tissue culture for generating same. This aspect of the present invention further provides a system for developing such a hybrid cotton plant which system includes cotton plants A-175 and R-208 or parts of thereof as a source of the breeding material.

According to another aspect of the present invention, there is provided a cotton plant designated INTERCOTT-34, representative seed of the cotton plant having been deposited under ATCC Patent Depository No.: PTA-4831.

As is illustrated in the Examples section which follows, INTERCOTT-34 has numerous important morphological and physiological characteristics including a narrow growth habit, short fruit branches, large, wide and dark-green leaves; tipped oval shaped boll, an average number of 4-5 locules per boll, averaging 125 days to reach 50 percent open bolls, an average number of 5 nodes before the first fruit branch, plant height of 90-100 cm at full maturity, medium pubescence of leaf and stem, presence of gossypol glands, presence of nectaries in flowers and leaves, light yellow petals, presence of flower petal spot, yellow flower pollen, seed index of 13.5 gram per 100 seeds, a light fuzzy seed coat, lint content of 37.5 per cent, fiber length average of 1.33 inches, fiber strength average of 34-36 gram per tex and fiber fineness of 3.8-4.0 micronaire, tolerance to Verticillium wilt, resistance to Fusarium wilt, and resistance to Alternaria leaf spot.

As is mentioned hereinabove, INTERCOTT-34 was generated by crossing female parental line A-34 and male parental line R-208. The characteristic traits of these parental lines are provided in Tables 1 and 2 of the Examples section that follows.

A comparison of INTERCOTT-34 to its parental lines, demonstrates that this interspecific hybrid exhibits several economically and agronomically advantageous traits over its respective parental lines. In particular, Verticillium wilt tolerance (compared with sensitive A-34), fiber length of 1.33 inches (compared with 1.14 and 1.25 inches of A-34 and R-208, respectively), fiber strength of 34-36 gram per tex (compared with 28.5 and 33 gram per tex of A-34 and R-208, respectively, and having short or light fuzzy seed coat enabling ginning by either roller or saw systems. In addition, INTERCOTT-34 has a restrained growth habit, concentrated crop and a high yield potential.

Seeds of the hybrid cotton plant of this aspect of the present invention can be generated using breeding and selection techniques as described hereinabove. Accordingly, seeds of the cotton plant INTERCOTT-34 were generated using a breeding process which began with the generation of highly specific parental lines A-34 and R-208, as described hereinabove and as is illustrated in FIG. 1 and in the Examples section which follows.

Once established, INTERCOTT-34 can be propagated from hybrid seeds or alternatively by using tissue culturing techniques, as described hereinabove. Thus, this aspect of the present invention provides novel hybrid cotton plants, seeds and tissue culture for generating same. This aspect of the present invention further provides a system for developing such a hybrid cotton plant which system includes cotton plants A-34 and R-208 or parts of thereof as a source of the breeding material.

According to another aspect of the present invention, there is provided a cotton plant designated INTERCOTT-145, representative seed of the cotton plant having been deposited under ATCC Patent Depository No.: PTA-4836.

As is illustrated in the Examples section which follows, INTERCOTT-145 has numerous important morphological and physiological characteristics including a medium-open growth habit, long fruit branches, medium, dark olive-green leaves; tipped oval shaped boll, an average number of 4-5 locules per boll, averaging 130 days to reach 50 percent open bolls, an average number of 6 nodes before the first fruit branch, plant height of 110-120 cm at fall maturity, high pubescence of leaf and stem, presence of gossypol glands, presence of nectaries in flowers and leaves, light yellow petals, presence of flower petal spot, yellow flower pollen, seed index of 12.5 gram per 100 seeds, a light fuzzy seed coat, lint content of 38 per cent, fiber length average of 1.34 inches, fiber strength average of 33-35 gram per tex and fiber fineness of 3.8-3.9 micronaire, resistance to Fusarium wilt, Verticillium wilt, Alternaria leaf spot, and tolerance to sucking pests such as Cicadellidae.

As is mentioned hereinabove, INTERCOTT-145 was generated by crossing female parental line A-14 and male parental line R-205. The characteristic traits of these parental lines are provided in Tables 1 and 2 of the Examples section that follows.

A comparison of INTERCOTT-145 to its parental lines, demonstrates that this interspecific hybrid exhibits several economically and agronomically advantageous traits over its respective parental lines. In particular, Verticillium wilt resistance (compared with sensitive A-14), fiber length of 1.34 inches compared with 1.13 and 1.29 inches of A-14 and R-205, respectively), fiber strength of 33-35 gram per tex (compared with 28.5 and 34 gram per tex of A-14 and R-205, respectively), fiber fineness of 3.8-3.9 micronaire (compared with 4.4 and 3.8 of A-14 and R-208, respectively), and having short or light fuzzy seed coat enabling ginning by either roller or saw systems. In addition, INTERCOTT-145 has an excellent recovery and is high yielding.

Seeds of the hybrid cotton plant of this aspect of the present invention can be generated using breeding and selection techniques as described hereinabove. Accordingly, seeds of the cotton plant INTERCOTT-145 were generated using a breeding process which began with the generation of highly specific parental lines A-14 and R-205, as described hereinabove and as is illustrated in FIG. 1 and in the Examples section which follows.

Once established, INTERCOTT-145 can be propagated from hybrid seeds or alternatively by using tissue culturing techniques, as described hereinabove. Thus, this aspect of the present invention provides novel hybrid cotton plants, seeds and tissue culture for generating same. This aspect of the present invention further provides a system for developing such a hybrid cotton plant which system includes cotton plants A-14 and R-205 or parts of thereof as a source of the breeding material.

According to another aspect of the present invention, there is provided a cotton plant designated INTERCOTT-83, representative seed of the cotton plant having been deposited under ATCC Patent Depository No.: PTA-4835.

As is illustrated in the Examples section which follows, INTERCOTT-83 has numerous important morphological and physiological characteristics including a medium growth habit, long fruit branches, medium, light green leaves; tipped oval shaped boll, an average number of 4-5 locules per boll, averaging 125 days to reach 50 percent open bolls, an average number of 6 nodes before the first fruit branch, plant height of 90-100 cm at full maturity, sparse pubescence of leaf and stem, presence of gossypol glands, presence of nectaries in flowers and leaves, light yellow petals, presence of flower petal spot, yellow flower pollen, seed index of 12.5 gram per 100 seeds, a naked seed coat, lint content of 37.5 per cent, fiber length average of 1.33 inches, fiber strength average of 32-34 gram per tex and fiber fineness of 3.5-3.6 micronaire, resistance to Fusarium wilt, resistance to Verticillium wilt and resistance to Alternaria leaf spot.

As is mentioned hereinabove, INTERCOTT-83 was generated by crossing female parental line A-83 and male parental line R-208. The characteristic traits of these parental lines are provided in Tables 1 and 2 of the Examples section that follows.

A comparison of INTERCOTT-83 to its parental lines, demonstrates that this interspecific hybrid exhibits several economically and agronomically advantageous traits over its respective parental lines. In particular, Verticillium wilt resistance (compared with sensitive A-83), fiber length of 1.33 inches (compared with 1.13 and 1.29 inches of A-83 and R-208, respectively), fiber strength of 32-34 gram per tex (compared with 27.5 and 33 gram per tex of A-83 and R-208, respectively), fiber fineness of 3.5-3.6 micronaire (compared with 4.1 and 3.7 micronaire of A-83 and R-208, respectively), and having naked seed coat that is particularly suitable for roller ginning. In addition, INTERCOTT-83 is high yielding and particularly adapted for short season requiring just 125 days to achieve maturity of 50 percent open boll.

Seeds of the hybrid cotton plant of this aspect of the present invention can be generated using breeding and selection techniques as described hereinabove. Accordingly, seeds of the cotton plant INTERCOTT-83 were generated using a breeding process which began with the generation of highly specific parental lines A-83 and R-208, as described hereinabove and as is illustrated in FIG. 1 and in the Examples section which follows.

Once established, INTERCOTT-83 can be propagated from hybrid seeds or alternatively by using tissue culturing techniques, as described hereinabove. Thus, this aspect of the present invention provides novel hybrid cotton plants, seeds and tissue culture for generating same. This aspect of the present invention further provides a system for developing such a hybrid cotton plant which system includes cotton plants A-83 and R-208 or parts of thereof as a source of the breeding material.

The interspecific hybrids of cotton disclosed herein have certain morphological and growth traits which differ from pure line varieties of either species and as such, these hybrid varieties are advantageous both economically and agronomically. Advantage of the interspecific hybrids of the present invention include superior adaptation to adverse growth conditions, resistant to pathogens, high yield, and superior lint quality.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include plant breeding and selection techniques. Such techniques are thoroughly explained in the literature. See, for example, Janick, J. (2001) Plant Breeding Reviews, John Wiley & Sons, is 252 p.; Jensen, N. F. ed. (1988) Plant Breeding Methodology, John Wiley & Soms, 676 p., Richard, A. J. ed. (1990) Plant Breeding Systems, Unwin Hyman, 529 p.; Walter, F. R. ed. (1987) Plant Breeding, Vol. I Theory and Techniques, Macmillan Pub. Co.; Slavko, B. ed. (1990) Principles and Methods of plant Breeding, Elsevier, 386 p.; and Allard, R. W. ed. (1999) Principles of Plant Breeding, John-Wiley & Sons, 240 p.; Cotton breeding techniques are described by Anon. (1977). Commercial Cotton Hybrids. The ICAC Recorder. Vol. XV no. 2: 3-14; and Davis D. D. (1978) Hybrid Cotton: Specific Problems and Potentials. Adv. Agron. 30: 129-1571; all of which are incorporated by reference as if fully set forth herein. Other general references are provided throughout this document. The procedures therein are believed to be well known in the art and are provided for the convenience of the reader. All the information contained therein is incorporated herein by reference.

### Breeding Process and Interspecific Hybrids Development

Germplasm collection: A rich and diversified cotton germplasm collection was established by collecting genotypes from various international sources. Gossypium barbadense germplasm was collected from China, Central Asia, Barbados island, USA and Peru. The genotypes were searched for traits including: early maturation, desired number of bolls per plant, resistance or tolerance to soil-borne diseases, adaptation to adverse climates and growth conditions, and exceptional fiber characteristics. G. hirsutum germplasm was collected from USA, India, China and Central Asia. The genotypes were searched for traits including: high yield potential, restrained growth, compatibility with G. barbadense, and resistance to plant diseases.

Agronomic growth conditions: Cotton plants were cultivated during the spring season in fields of clay loam soil located at the coastal valley of Israel. The plots were drip or sprinkler irrigated with 250-300 mm of water during the course of the growth season. N,P,K fertilizers were added according to soil analysis and pesticides were administered when required to maintain healthy crops. Plant height was restrained by applying growth regulator Mepiquat Chloride (Pix) at early or late stages with 300-1500 ml per hectare.

Parent lines selection: Selected genotypes were planted in a “Breeding Garden” where hybridizations took place. Two, three and four way crosses were made mainly between genotypes within each species to produce approximately 200 segregating F2 populations. Pedigree selections started in F2 populations which showed promising genetic variation and carried out for 6 to 8 consecutive selfing generations until achieving an acceptable homozygosity. Cross-species hybridizations were made using the G. hirsutum lines as female parents while the G. babadense lines served as male parents. The F1 plants were then evaluated based on basic parameters including: number and size of bolls, plant height, lint yield, lint percent, and fiber characteristics (length, strength and fineness). Based on the best performing F1 crosses, about 15 lines of G. barbadense (males) and 50 lines of G. hirsutum (females) were selected for conducting crosses of most of the combinations between male and female parents. Accordingly, pure lines having the superior combining ability and seed setting were selected.

Introduction of male sterility: The selected pure lines were provided with the male-sterility system using the germplasm lines according to Meyer (1973) [Registration of sixteen germplasm lines of Upland cotton. Crop Sci. 13: 778].

Accordingly, selected G. hirsutum lines were backcrossed, with the breeding line DES HAMS-16 to become male sterile (MS) female parents. The MS lines were designated “A” lines, while the recurrent original fertile lines became the maintainers of their MS lines respectively, designated as “B” lines. Selected pure breeding lines of G. hirsutum chosen for further breeding were designated A/B-195, A/B-151, A/B-34, A/B-83 and A/B-14. The traits of these selected G. hirsutum female lines are described in Table 1 bellow.

TABLE 1
Description of Gossypium hirsutum parent lines

| | Gossypium hirsutum (Upland) “A/B” lines | | | | | |
|---|---|---|---|---|---|---|
| Trait | # 195 | # 175 | # 83 | # 151 | # 34 | # 14 |
| Plant type | erect | open | normal | open | narrow | normal |
| Fruit branches | medium | short | medium | medium | V. short | medium |
| (Length) | | | | | | |
| Leaf type | normal | medium | medium | small | large | medium |
| (size) | | | | | | |
| Leaf color | dark | green | Light | dark | dark | normal |
| (green) | | | | | | |
| Boll shape | oval | rounded | Oval | rounded | rounded | rounded |
| No. locules/ | 4-5 | 4 | 4 | 4 | 4-5 | 4 |
| boll | | | | | | |
| Maturity- | 130 | 100 | 110 | 120 | 120 | 120 |
| days to | | | | | | |
| 50% open boll | | | | | | |
| Node no. | 5 | 4 | 4.5 | 5 | 4.5 | 5 |
| of 1^{st} | | | | | | |
| fruit | | | | | | |
| branch | | | | | | |
| Plant height | 120 | 90 | 110 | 110 | 100 | 110 |
| cm. (at full | | | | | | |
| maturity) | | | | | | |
| Pubescence of | medium | medium | Sparse | sparse | medium | sparse |
| leaf & stem | | | | | | |
| Gossypol | present | present | present | present | present | present |
| Glands | | | | | | |
| Nectaries | present | present | present | present | present | present |
| Flower petals | cream | cream | cream | cream | cream | cream |
| Flower petal | absent | absent | absent | absent | absent | absent |
| spot | | | | | | |
| Flower pollen | cream | cream | cream | cream | cream | cream |
| Seed Index | 12 | 10.5 | 11.5 | 11 | 12.5 | 11.5 |
| (gr/100 seeds) | | | | | | |
| Seed coat | fuzzy | fuzzy | Fuzzy | fuzzy | fuzzy | fuzzy |
| | | | less | | | |
| Lint percent | 36.5 | 36 | 37 | 37 | 37.5 | 38.5 |
| Seed cotton | 6.5 | 5.0 | 5.5 | 6.0 | 7.0 | 6.0 |
| (gr/boll) | | | | | | |
| Fiber length | 1.15 | 1.12 | 1.13 | 1.13 | 1.14 | 1.13 |
| (inches) | | | | | | |
| Fiber | 27.5 | 27.5 | 27.5 | 28 | 28.5 | 28.5 |
| Strength | | | | | | |
| (gr/tex) | | | | | | |
| Fineness | 4.3 | 4.1 | 4.1 | 4.4 | 4.3 | 4.5 |
| (Micronaire) | | | | | | |
| Fusarium wilt | resistant | resistant | resistant | resistant | resistant | resistant |
| Verticillium | no | no | no | no | no | no |
| wilt | | | | | | |
| resistance | | | | | | |

Selected G. barbadense lines were backcrossed with the breeding line DES HAF-16 to incorporate the dominant gene for fertility restoration. Lines which showed the capacity to restore fertility were selected as restorer male parents, and designated as “R” lines. The selected pure breeding lines of G. barbadense were designated R-208 and R-205. The traits of these selected G. barbanense restores male lines are described in Table 2 bellow.

TABLE 2
Description of Gossypium barbadense parent lines

Gossypium barbadense
(Pima)

| Trait | R-208 | R-205 |
|---|---|---|
| Plant type | open | normal |
| Fruit branches | long | medium |
| (Length) | | |
| Leaf type (size) | normal | Small |
| Leaf color | Light | Olive |
| (green) | | |
| Boll shape | elongated | Elongate |
| No. locules/boll | 3 | 3 |
| Maturity- days to | 140 | 150 |
| 50% open boll | | |
| Node no. of 1^{st} | 6 | 6.5 |
| fruit branch | | |
| Plant height cm. | 90 | 100 |
| (at full maturity) | | |
| Pubescence of | sparse | High |
| leaf & stem | | |
| Gossypol Glands | present | present |
| Nectaries | present | present |
| Flower petals | yellow | yellow |
| Flower petal spot | present | present |
| Flower pollen | yellow | yellow |
| Seed Index | 10 | 10.5 |
| (gr/100 seeds) | | |
| Seed coat | Fuzzy less | naked |
| Lint percent | 35 | 36 |
| Seed cotton | 3.0 | 3.5 |
| (gr/boll) | | |
| Fiber length | 1.25 | 1.29 |
| (inches) | | |
| Fiber Strength | 33 | 34 |
| (gr/tex) | | |
| Fineness | 3.7 | 3.8 |
| (Micronaire) | | |
| Fusarium wilt | resistant | tolerant |
| Verticillium wilt | resistant | resistant |
| resistance | | |

Interspecific hybrids development: Hybridizations were made between the “A” and “R” parental lines under field conditions using honeybees as pollinators. This operation enabled increasing production of F1 hybrid seeds. The hybrid performance was evaluated in field trials carried out during 2-3 consecutive seasons at several locations. Hybrids exhibiting superior yield and lint quality were selected for additional regional field trials carried under variable climatic and growing conditions, including conditions of drought, salinity, short season, excessive cloudiness, low temperature and infestation with pathogens. The final selection of hybrids was based on high lint quality, higher yield, concentrated yield, and tolerance to adverse climatic and growing conditions.

### Interspecific Hybrids Description

The selected interspecific hybrids of the present invention were designated INTERCOTT-35, INTERCOTT-51, INTERCOTT-75, INTERCOTT-34, INTERCOTT-145 and INTERCOTT-83. All selected hybrids exhibit agronomic advantages over non-hybrid varieties and share the following traits:
(a) a rapid seedling emergence and early growth rate, even at low temperature;
(b) a high water-use efficiency;
(c) a high tolerance to plant pathogens, e.g., as Verticilium Spp. and Fussarium Spp., and Alternaria Spp.;
(d) the number and size of flower bud (square) is similar to that typically found in G. barbadense;
(e) the structure and size of flower is similar to that typically found in G. barbadense;
(f) the flower is large and corolla color is light yellow;
(g) the shape of boll is similar to that typically found in G. barbadense but larger;
(h) the boll is comprised of 4-5 cells similarly to G. hirsutum;
(i) the size of boll is similar to that typically found in G. hirsutum; and
(j) the seed coat is covered by a short or light fuzz facilitating ginning by both roller and saw systems.

The selected hybrids are individually characterized by traits such as an adaptation to specific climatic or growing conditions, duration of growth cycle, yield potential, and fiber/lint quality characteristic values. The respective parental lines and traits of the selected hybrids are summarized in Tables 3a and 3b bellow.

TABLE 3a
Description of Cotton Hybrids

| | Hybrid name | | |
|---|---|---|---|
| | INTER- | INTER- | INTER- |
| | COTT-35 | COTT-51 | COTT-75 |
| Male parent | R - 208 | R - 208 | R - 208 |
| Female parent | A - 195 | A - 151 | A - 175 |
| Plant type | Semi erect | Medium open | Short open |
| Fruit branches | Long | Long | medium |
| Leaf type | Large | Large | medium |
| Leaf color | Dark green | light green | Light green |
| Boll shape | Tipped Oval | Tipped Oval | Tipped Oval |
| No. locules/boll | 4-5 | 4-5 | 4 |
| Maturity- days | 140 | 130 | 120 |
| to 50% open boll | | | |
| No. node of 1^{st} | 6 | 5.5 | 5 |
| fruit branch | | | |
| Plant height cm. | 110-130 | 100-110 | 90-100 |
| (at full maturity) | | | |
| Pubescence of | Medium | Sparse | medium |
| Leaf & stem | | | |
| Gossypol Glands | Present | Present | present |
| Nectaries - floral | Present | Present | present |
| & leaf | | | |
| Flower petals | Yellowish | Yellowish | yellowish |
| Flower petal spot | Present | Present | present |
| Flower pollen | Yellow | Yellow | yellow |
| Seed index- g/100 | 13.0 | 12.5 | 12.0 |
| Seed coat | Light fuzzy | Light fuzzy | Light fuzzy |
| Lint percent | 37 | 36.5 | 36 |
| Seed cotton g/boll | 4.5 | 4.5 | 4 |
| Fiber length inch. | 1.34 | 1.33 | 1.32 |
| Fiber strength. g/tex | 36-38 | 32-34 | 31-33 |
| Fineness (mic.) | 3.7-3.9 | 3.7-3.9 | 3.8-3.9 |
| Fusarium wilt | Resistant | Resistant | Resistant |
| Verticillium wilt | Resistant | Resistant | Resistant |
| Alternaria leaf sp. | Resistant | Resistant | Resistant |
| Adaptation to/ | To drought & | For Medium | For Short |
| recommendations | salinity | season | season |
| Expected output - | High yield | Concentrate | Early |
| In addition to high | under Stress | yield under | flowering & |
| lint quality | conditions | limited irrigation | maturation |

TABLE 3b
Description of Cotton Hybrids

| | Hybrid name | | |
|---|---|---|---|
| | INTER- | INTER- | INTER- |
| | COTT-34 | COTT-145 | COTT-83 |
| Male parent | R - 208 | R - 205 | R - 208 |
| Female parent | A - 34 | A - 14 | A - 83 |
| Plant type | Narrow | Medium open | medium |
| Fruit branches | Short | Long | long |
| Leaf type | Large wide | Medium | medium |
| Leaf color | Dark green | Dark olive | Light green |
| Boll shape | Tipped Oval | Tipped Oval | Tipped Oval |
| No. locules/boll | 4-5 | 4 | 4-5 |
| Maturity- days | 125 | 130 | 125 |
| to 50% open boll | | | |
| No. node of 1^{st} | 5 | 6 | 6 |
| fruit branch | | | |
| Plant height cm. | 90-100 | 110-120 | 90-100 |
| (at full maturity) | | | |
| Pubescence of | Medium | High | Sparse |
| leaf & stem | | | |
| Gossypol Glands | Present | Present | Present |
| Nectaries - floral | Present | Present | Present |
| & leaf | | | |
| Flower petals | Yellowish | Yellowish | yellowish |
| Flower petal spot | Present | Present | present |
| Flower pollen | Yellow | Yellow | yellow |
| Seed index- g/100 | 13.5 | 12.5 | 12.5 |
| Seed coat | Light fuzzy | Light fuzzy | naked |
| Lint percent | 37.5 | 38 | 37.5 |
| Seed cotton g/boll | 5 | 5 | 4.5 |
| Fiber length inch. | 1.33 | 1.34 | 1.33 |
| Fiber strength. g/tex | 34-36 | 33-35 | 32-34 |
| Fineness (mic.) | 3.8-4.0 | 3.8-3.9 | 3.5-3.6 |
| Fusarium wilt | Resistant | Resistant | Resistant |
| Verticillium wilt | Tolerant | Resistant | Resistant |
| Alternaria leaf sp. | Resistant | Resistant | Resistant |
| Adaptation to/ | Restrained | Medium-short | Partly |
| recommendations | growth | season | irrigation |
| Expected output - | High, | Excellent | High yield in |
| In addition to high | concentrate | recovery; High | short season; |
| lint quality | yield | yield; tolerance | particularly |
| | | to insects such as | suitable for |
| | | Jussids | roller ginning |
| | | (Cicadellidae) | |

As is demonstrated by Table 3a, hybrid INTERCOTT-35 exhibits high yield potential under stress conditions, adaptation to drought, adaptation to salinity, resistance to Fusarium wilt, resistance to Verticillium wilt, resistance to Alternaria leaf spot, and high lint quality; hybrid INTERCOTT-51 demonstrates concentrated yield under limited irrigation, superiority for medium season, resistance to Fusarium wilt, resistance to Verticillium wilt, resistance to Alternaria leaf spot, and high lint quality; while hybrid INTERCOTT-75 demonstrates early flowering and maturation, resistance to Fusarium wilt, resistance to Verticillium wilt, resistance to Alternaria leaf spot, and high lint quality.

As is demonstrated by Table 3b, hybrid INTERCOTT-34 demonstrates high yield potential, concentrated yield, resistance to Fusarium wilt, resistance to Verticillium wilt, resistance to Alternaria leaf spot, and high lint quality; hybrid INTERCOTT-145 demonstrates a unique combination of hairy leaf and stem surfaces which contribute to high tolerance to sucking insects such as “Jussids” (Cicadellidae), high yield potential, excellent recovery, resistance to Fusarium wilt, resistance to Verticillium wilt, resistance to Alternaria leaf spot, and high lint quality; while hybrid INTERCOTT-83 demonstrates high yield potential in short season, resistance to Fusarium wilt, resistance to Verticillium wilt, resistance to Alternaria leaf spot, and high lint quality, in particular following roller ginning.

In summary, the interspecific hybrids generated by the present invention exhibit morphological and growth traits distinct from pure line varieties of either species. The selected interspecific hybrids are characterized by being superiorly adapted to adverse growth conditions, such as drought or salinity stress; by having high fiber yield and quality potential; and by enabling fiber separation by both roller and saw ginning.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, and patent applications mentioned in this specification are herein incorporated in their entirety by reference into the specification, to the same extent as if each individual publication, patent, or patent application was specifically and individually indicated to be incorporated herein by reference. In addition, citation or identification of any reference in this application shall not be construed as an admission that such reference is available as prior art to the present invention.

### Deposit Information

Propagating material of the cotton plant varieties of the present invention is maintained by American Type Culture Collection (Manassas, Va. 20110) since Dec. 2, 2002 under the following depository numbers: INTERCOTT-35—ATCC PTA-4832; INTERCOTT-51—ATCC PTA-4833; INTERCOTT-75—ATCC PTA-4834; INTERCOTT-34—ATCC PTA-4831; INTERCOTT-145—ATCC PTA-4836; and INTERCOTT-83—ATCC PTA-4835.

What is claimed is:

1. A hybrid cotton plant designated INTERCOTT-35, representative seed of said hybrid cotton plant having been deposited under ATCC Patent Depository No.: PTA-4831.

2. Seed of said hybrid cotton plant of claim 1.

3. A tissue culture of regenerable cells of said hybrid cotton plant of claim 1.

4. The tissue culture of claim 3, wherein the tissue culture regenerates plants capable of expressing all the morphological and physiological characteristics of said hybrid cotton plant.

5. A tissue culture of claim 4, wherein said tissue culture is regenerated from cells or protoplasts of a tissue selected from the group consisting of seeds, leaves, stems, pollens, roots, root tips, anthers, ovules, petals, flowers, embryos, fibers and bolls.

6. A cotton plant, or its parts, wherein at least one ancestor of said hybrid cotton plant is the cotton plant INTERCOTT-35, said cotton plant capable of expressing at least one trait selected from the group consisting of a tolerance to suboptimal water supply, a tolerance to salinity, a tolerance to suboptimal temperature, a tolerance to suboptimal light, a resistance to Fusarium wilt, a resistance to Verticillium wilt, a resistance to Alternaria leaf spot, an average period of 140 days to 50 percent open boll, a fiber length average not lower than 1.34 inches, a filer strength average not lower than 36 gram per tex, a fiber fineness average not higher than 3.9 micronaire, tipped oval shaped boll, an average number of 4-5 locules per boll and light yellow petals.

7. A method of developing a hybrid cotton plant using plant breeding techniques which employ a cotton plant, or its parts, as a source of plant breeding material, the method comprising utilizing cotton plants Gossypium hirsutum designated line A-195, and Gossypium barbadense designated line R-208 as a source of breeding material.

8. The method of claim 7, wherein the plant breeding techniques are selected from the group consisting of recurrent selection, backcrossing, pedigree breeding, restriction fragment length polymorphism enhanced selection, genetic marker enhanced selection, and transformation.

9. A system for developing a hybrid cotton plant using plant breeding techniques, the system comprising cotton plants A-195 and R-208 or parts of said cotton plants as a source of the breeding material.

10. The system of claim 9, wherein at least one of said cotton plants is male sterile.

11. A cotton plant characterized by a combination of traits leading to a commercial yield higher than at least one parent of the cotton plant under growth conditions, selected from the group consisting of suboptimal water supply, suboptimal salinity, suboptimal temperature, suboptimal light, a growth cycle period shorter than 170 days, and infestation of pathogens.

12. The cotton plant of claim 11, wherein said pathogens are selected from the group consisting of Verticillium spp., Fusarium spp., and Alternaria spp.

13. The cotton plant of claim 11, wherein the cotton plant is hybrid cotton plant INTERCOTT-35 and its parents are cotton plants A-195 and R-208.

14. A planted field comprising cotton plants A-195 and R-208.

15. The planted field of claim 14, wherein at least one of said cotton plants is male sterile.

16. The planted field of claim 14, wherein the field is characterized by a planting pattern enabling cross pollination between cotton plants A-195 and R-208.

17. A planted field comprising cotton plants B-195 and R-208.

18. A hybrid cotton plant designated INTERCOTT-51, representative seed of said hybrid cotton plant having been deposited under ATCC Patent Depository No.: PTA-4833.

19. Seed of said hybrid cotton plant of claim 18.

20. A tissue culture of regenerable cells of said hybrid cotton plant of claim 18.

21. The tissue culture of claim 20, wherein the tissue culture regenerates plants capable of expressing all the morphological and physiological characteristics of said hybrid cotton plant.

22. A tissue culture of claim 21, wherein said tissue culture is regenerated from cells or protoplasts of a tissue selected from the group consisting of seeds, leaves, stems, pollens, roots, root tips, anthers, ovules, petals, flowers, embryos, fibers and bolls.

23. A cotton plant, or its parts, wherein at least one ancestor of said hybrid cotton plant is the cotton plant INTERCOTT-5 1, said cotton plant capable of expressing at least one trait selected from the group consisting of a tolerance to suboptimal water supply, a tolerance to salinity, a tolerance to suboptimal temperature, a tolerance to suboptimal light, a resistance to Fusarium wilt, a resistance to Verticillium wilt, a resistance to Alternaria leaf spot, an average period of 130 days to 50 percent open boll, a fiber length average not lower than 1.33 inches, a fiber strength average not lower than 34 gram per tex, a fiber fineness average not higher than 3.9 micronaire. tipped oval shaped boll, an average number of 4-5 locules per boll and light yellow petals.

24. A method of developing a hybrid cotton plant using plant breeding techniques which employ a cotton plant, or its parts, as a source of plant breeding material, the method comprising utilizing cotton plants Gossypium hirsutum designated line A-151, and Gossypium barbadense designated line R-208 as a source of breeding material.

25. The method of claim 24, wherein the plant breeding techniques are selected from the group consisting of recurrent selection, backcrossing, pedigree breeding, restriction fragment length polymorphism enhanced selection, genetic marker enhanced selection, and transformation.

26. A system for developing a hybrid cotton plant using plant breeding techniques, the system comprising cotton plants A-151 and R-208 or parts of said cotton plants as a source of the breeding material.

27. The system of claim 26, wherein at least one of said cotton plants is male sterile.

28. A cotton plant characterized by a combination of traits leading to a commercial yield higher than at least one parent of the cotton plant under growth conditions, selected from the group consisting of suboptimal water supply, suboptimal salinity, suboptimal temperature, suboptimal light, a growth cycle period shorter than 160 days, and infestation of pathogens.

29. The cotton plant of claim 28, wherein said pathogens are selected from the group consisting of Verticillium spp., Fusarium spp., and Alternaria spp.

30. The cotton plant of claim 28, wherein the cotton plant is hybrid cotton plant INTERCOTT-51 and its parents are cotton plants A-151 and R-208.

31. A planted field comprising cotton plants A-151 and R-208.

32. The planted field of claim 31, wherein at least one of said cotton plants is male sterile.

33. The planted field of claim 31, wherein the field is characterized by a planting pattern enabling cross pollination between cotton plants A-151 and R-208.

34. A planted field comprising cotton plants B-151 and R-208.

35. A hybrid cotton plant designated INTERCOTT-75, representative seed of said hybrid cotton plant having been deposited under ATCC Patent Depository No.: PTA-4834.

36. Seed of said hybrid cotton plant of claim 35.

37. A tissue culture of regenerable cells of said hybrid cotton plant of claim 35.

38. The tissue culture of claim 37, wherein the tissue culture regenerates plants capable of expressing all the morphological and physiological characteristics of said hybrid cotton plant.

39. A tissue culture of claim 38, wherein said tissue culture is regenerated from cells or protoplasts of a tissue selected from the group consisting of seeds, leaves, stems, pollens, roots, root tips, anthers, ovules, petals, flowers, embryos, fibers and bolls.

40. A cotton plant, or its parts, wherein at least one ancestor of said hybrid cotton plant is the cotton plant INTERCOTT-75, said cotton plant capable of expressing at least one trait selected from the group consisting of a tolerance to suboptimal water supply, a tolerance to salinity, a tolerance to suboptimal temperature, a tolerance to suboptimal light, a resistance to Fusarium wilt, a resistance to Verticillium wilt, a resistance to Alternaria leaf spot, an average period of 120 days to 50 percent open boll, a fiber length average not lower than 1.32 inches, a fiber strength average not lower than 33 gram per tex, a fiber fineness average not higher than 3.9 micronaire, tipped oval shaped boll, an average number of 4-5 locules per boll and light yellow petals.

41. A method of developing a hybrid cotton plant using plant breeding techniques which employ a cotton plant, or its parts, as a source of plant breeding material, the method comprising utilizing cotton plants Gossypium hirsutum designated line A-175 ATCC, and Gossypium barbadense designated line R-208 as a source of breeding material.

42. The method of claim 41, wherein the plant breeding techniques are selected from the group consisting of recurrent selection, backcrossing, pedigree breeding, restriction fragment length polymorphism enhanced selection, genetic marker enhanced selection, and transformation.

43. A system for developing a hybrid cotton plant using plant breeding techniques, the system comprising cotton plants A-175 and R-208 or parts of said cotton plants as a source of the breeding material.

44. The system of claim 43, wherein at least one of said cotton plants is male sterile.

45. A cotton plant characterized by a combination of traits leading to a commercial yield higher than at least one parent of the cotton plant under growth conditions, selected from the group consisting of suboptimal water supply, suboptimal salinity, suboptimal temperature, suboptimal light, a growth cycle period shorter than 150 days, and infestation of pathogens.

46. The cotton plant of claim 45, wherein said pathogens are selected from the group consisting of Verticillium spp., Fusarium spp., and Alternaria spp.

47. The cotton plant of claim 45, wherein the cotton plant is hybrid cotton plant INTERCOTT-75 and its parents are cotton plants A-175 and R-208.

48. A planted field comprising cotton plants A-175 and R-208.

49. The planted field of claim 48, wherein at least one of said cotton plants is male sterile.

50. The planted field of claim 48, wherein the field is characterized by a planting pattern enabling cross pollination between cotton plants A-175 and R-208.

51. A planted field comprising cotton plants B-175 and R-208.

52. A hybrid cotton plant designated INTERCOTT-34, representative seed of said hybrid cotton plant having been deposited under ATCC Patent Depository No.: PTA-4831.

53. Seed of said hybrid cotton plant of claim 52.

54. A tissue culture of regenerable cells of said hybrid cotton plant of claim 52.

55. The tissue culture of claim 54, wherein the tissue culture regenerates plants capable of expressing all the morphological and physiological characteristics of said hybrid cotton plant.

56. A tissue culture of claim 55, wherein said tissue culture is regenerated from cells or protoplasts of a tissue selected from the group consisting of seeds, leaves, stems, pollens, roots, root tips, anthers, ovules, petals, flowers, embryos, fibers and bolls.

57. A cotton plant, or its parts, wherein at least one ancestor of said hybrid cotton plant is the cotton plant INTERCOTT-34, said cotton plant capable of expressing at least one trait selected from the group consisting of a tolerance to suboptimal water supply, a tolerance to salinity, a tolerance to suboptimal temperature, a tolerance to suboptimal light, a resistance to Fusarium wilt, a resistance to Verticillium wilt, a resistance to Alternaria leaf spot, an average period of 125 days to 50 percent open boll, a fiber length average not lower than 1.33 inches, a fiber strength average not lower than 36 gram per tex, a fiber fineness average not higher than 4.0 micronaire, tipped oval shaped boll, an average number of 4-5 locules per boll and light yellow petals.

58. A method of developing a hybrid cotton plant using plant breeding techniques which employ a cotton plant, or its parts, as a source of plant breeding material, the method comprising utilizing cotton plants Gossypium hirsutum designated line A-34, and Gossypium barbadense designated line R-208 as a source of breeding material.

59. The method of claim 58, wherein the plant breeding techniques are selected from the group consisting of recurrent selection, backcrossing, pedigree breeding, restriction fragment length polymorphism enhanced selection, genetic marker enhanced selection, and transformation.

60. A system for developing a hybrid cotton plant using plant breeding techniques, the system comprising cotton plants A-34 and R-208 or parts of said cotton plants as a source of the breeding material.

61. The system of claim 60, wherein at least one of said cotton plants is male sterile.

62. A cotton plant characterized by a combination of traits leading to a commercial yield higher than at least one parent of the cotton plant under growth conditions, selected from the group consisting of suboptimal water supply, suboptimal salinity, suboptimal temperature, suboptimal light, a growth cycle period shorter than 160 days, and infestation of pathogens.

63. The cotton plant of claim 62, wherein said pathogens are selected from the group consisting of Verticillium spp., Fusarium spp., and Alternaria spp.

64. The cotton plant of claim 62, wherein the cotton plant is hybrid cotton plant INTERCOTT-34 and its parents are cotton plants A-34 and R-208.

65. A planted field comprising cotton plants A-34 and R-208.

66. The planted field of claim 65, wherein at least one of said cotton plants is male sterile.

67. The planted field of claim 65, wherein the field is characterized by a planting pattern enabling cross pollination between cotton plants A-34 and R-208.

68. A planted field comprising cotton plants B-34 and R-208.

69. A hybrid cotton plant designated INTERCOTT-145, representative seed of said hybrid cotton plant having been deposited under ATCC Patent Depository No.: PTA-4836.

70. Seed of said hybrid cotton plant of claim 69.

71. A tissue culture of regenerable cells of said hybrid cotton plant of claim 69.

72. The tissue culture of claim 71, wherein the tissue culture regenerates plants capable of expressing all the morphological and physiological characteristics of said hybrid cotton plant.

73. A tissue culture of claim 72, wherein said tissue culture is regenerated from cells or protoplasts of a tissue selected from the group consisting of seeds, leaves, stems, pollens, roots, root tips, anthers, ovules, petals, flowers, embryos, fibers and bolls.

74. A cotton plant, or its parts, wherein at least one ancestor of said hybrid cotton plant is the cotton plant INTERCOTT-145, said cotton plant capable of expressing at least one trait selected from the group consisting of a tolerance to suboptimal water supply, a tolerance to salinity, a tolerance to suboptimal temperature, a tolerance to suboptimal light, a resistance to Fusarium wilt, a resistance to Verticillium wilt, a resistance to Alternaria leaf spot, tolerant to Cicadellidae insects, an average period of 130 days to 50 percent open boll, a fiber length average not lower than 1.34 inches, a fiber strength average not lower than 35 gram per tex, a fiber fineness average not higher than 3.9 micronaire, tipped oval shaped boll, an average number of 4-5 locules per boll and light yellow petals.

75. A method of developing a hybrid cotton plant using plant breeding techniques which employ a cotton plant, or its parts, as a source of plant breeding material, the method comprising utilizing cotton plants Gossypium hirsutum designated line A-14, and Gossypium barbadense designated line R-205 as a source of breeding material.

76. The method of claim 75, wherein the plant breeding techniques are selected from the group consisting of recurrent selection, backcrossing, pedigree breeding, restriction fragment length polymorphism enhanced selection, genetic marker enhanced selection, and transformation.

77. A system for developing a hybrid cotton plant using plant breeding techniques, the system comprising cotton plants A-14 and R-205 or parts of said cotton plants as a source of the breeding material.

78. The system of claim 77, wherein at least one of said cotton plants is male sterile.

79. The system of claim 77, wherein at least one of said cotton plants exhibits a hair cover on leaves and stems of a density higher than that of an Acala type cotton plant.

80. A cotton plant characterized by a combination of traits leading to a commercial yield higher than at least one parent of the cotton plant under growth conditions, selected from the group consisting of suboptimal water supply, suboptimal salinity, suboptimal temperature, suboptimal light, a growth cycle, period shorter than 160 days, and infestation of pathogens.

81. The cotton plant of claim 80, wherein said pathogens are selected from the group consisting of Verticillium spp., Fusarium spp., Alternaria spp and Cicadellidae spp.

82. The cotton plant of claim 80, wherein the cotton plant is hybrid cotton plant INTERCOTT-145 and its parents are cotton plants A-14 and R-205.

83. A planted field comprising cotton plants A-14 and R-205.

84. The planted field of claim 83, wherein at least one of said cotton plants is male sterile.

85. The planted field of claim 83, wherein the field is characterized by a planting pattern enabling cross pollination between cotton plants A-14 and R-205.

86. A planted field comprising cotton plants B-14 and R-205.

87. A hybrid cotton plant designated INTERCOTT-83, representative seed of said hybrid cotton plant having been deposited under ATCC Patent Depository No.: PTA-4835.

88. Seed of said hybrid cotton plant of claim 87.

89. A tissue culture of regenerable cells of said hybrid cotton plant of claim 87.

90. The tissue culture of claim 89, wherein the tissue culture regenerates plants capable of expressing all the morphological and physiological characteristics of said hybrid cotton plant.

91. A tissue culture of claim 90, wherein said tissue culture is regenerated from cells or protoplasts of a tissue selected from the group consisting of seeds, leaves, stems, pollens, roots, root tips, anthers, ovules, petals, flowers, embryos, fibers and bolls.

92. A cotton plant, or its parts, wherein at least one ancestor of said hybrid cotton plant is the cotton plant INTERCOTT-83, said cotton plant capable of expressing at least one trait selected from the group consisting of a tolerance to suboptimal water supply, a tolerance to salinity, a tolerance to suboptimal temperature, a tolerance to suboptimal light, a resistance to Fusarium wilt, a resistance to Verticillium wilt, a resistance to Alternaria leaf spot, an average period of 125 days to 50 percent open boll, a fiber length average not lower than 1.33 inches, a fiber strength average not lower than 34 gram per tex, a fiber fineness average not higher than 3.6 micronaire, tipped oval shaped boll, an average number of 4-5 locules per boll and light yellow petals.

93. A method of developing a hybrid cotton plant using plant breeding techniques which employ a cotton plant, or its parts, as a source of plant breeding material, the method comprising utilizing cotton plants Gossypium hirsutum designated line A-83, and Gossypium barbadense designated line R-208 as a source of breeding material.

94. The method of claim 93, wherein the plant breeding techniques are selected from the group consisting of recurrent selection, backcrossing, pedigree breeding, restriction fragment length polymorphism enhanced selection, genetic marker enhanced selection, and transformation.

95. A system for developing a hybrid cotton plant using plant breeding techniques, the system comprising cotton plants A-83 and R-208 or parts of said cotton plants as a source of the breeding material.

96. The system of claim 95, wherein at least one of said cotton plants is male sterile.

97. A cotton plant characterized by a combination of traits leading to a commercial yield higher than at least one parent of the cotton plant under growth conditions, selected from the group consisting of suboptimal water supply, suboptimal salinity, suboptimal temperature, suboptimal light, a growth cycle period shorter than 160 days, and infestation of pathogens.

98. The cotton plant of claim 97, further characterized by a naked Pima type seed coat.

99. The cotton plant of claim 97, wherein said pathogens are selected from the group consisting of Verticillium spp., Fusarium spp., and Alternaria spp.

100. The cotton plant of claim 97, wherein the cotton plant is hybrid cotton plant INTERCOTT-83 and its parents are cotton plants A-83 and R-208.

101. A planted field comprising cotton plants A-83 and R-208.

102. The planted field of claim 101, wherein at least one of said cotton plants is male sterile.

103. The planted field of claim 101, wherein the field is characterized by a planting pattern enabling cross pollination between cotton plants A-83 and R-208.

104. A planted field comprising cotton plants B-83 and R-208.

105. A method of developing a hybrid cotton plant using plant breeding techniques, the method comprising utilizing a first cotton plant selected from the group consisting of A-195, A-151, A-175, A-34, A-14 and A-83, and a second cotton plant selected from the group consisting of R-205 and R-208, as sources of breeding material.

106. A system for developing a hybrid cotton plant using plant breeding techniques, the system comprising utilizing a first cotton plant selected from the group consisting of A-195, A-151, A-175, A-34, A-14 and A-83, and a second cotton plant selected from the group consisting of R-205 and R-208, as sources of breeding material.
